(19) United States
(12) Patent Application Publication
(10) Pub. No.: US 2025/0207158 A1
(43) Pub. Date: Jun. 26, 2025

(54) FERMENTATION PROCESS FOR THE PRODUCTION OF LIPIDS FROM OLEAGINOUS YEASTS

(71) Applicant: VERSALIS S.P.A., San Donato Milanese (MI) (IT)

(72) Inventors: Daniela CUCCHETTI, Novara (NO) (IT); Alessandra FRATTINI, Fraz. Rivalta Scrivia, Tortona (AL) (IT); Alessandro DEL SEPPIA, Mantova (MN) (IT); Tommaso PRANDO, Novara (NO) (IT)

(21) Appl. No.: 18/849,855

(22) PCT Filed: Mar. 21, 2023
(86) PCT No.: PCT/IB2023/052761

§ 371 Date: Sep. 23, 2024

(30) Foreign Application Priority Data
Mar. 24, 2022 (IT) 102022000005897

(51) Int. Cl. C12P 7/64; C12N 1/16

(52) U.S. Cl. CPC C12P 7/64; C12N 1/16

(57) ABSTRACT

A fermentation process for the production of lipids in the presence of at least one oleaginous yeast includes the steps of:
growing the oleaginous cell biomass wherein the respiratory quotient of the at least one oleaginous yeast is kept at a constant value between 0.85 and 1.2, preferably between 0.9 and 1.15, and more preferably equal to 1.1; and a further
step of lipid production wherein the respiratory quotient of the at least one oleaginous yeast is kept at a constant value between 1.2 and 2.6, preferably between 1.3 and 2.55, and more preferably equal to 1.4.
The lipids thus obtained are advantageously used in the production of biofuels which can be used as such, or mixed with other fuels in diesel engines for automotive or aviation.

### CROSS-REFERENCE TO RELATED APPLICATIONS

This application is a 35 U.S.C. § 371 National Stage patent application of PCT/IB2023/052761, filed on 21 Mar. 2023, which claims the benefit of Italian patent application 102022000005897, filed on 24 Mar. 2022, the disclosures of which are incorporated herein by reference in their entirety.

### TECHNICAL FIELD

The present disclosure relates to a fermentation process for the production of lipids from oleaginous yeasts.

More particularly, the present disclosure relates to a fermentation process for the production of lipids in the presence of at least one oleaginous yeast comprising: a first step of growth of the oleaginous cell biomass and a second step of lipid production, wherein the respiratory quotient (RQ) of said at least one oleaginous yeast is maintained at specific values, said values being different for said first step of growth of the oleaginous cell biomass and for said second step of lipid production and being kept constant during said first step and said second step.

The lipids thus obtained can be advantageously used in the production of biofuels which can be used as such, or mixed with other fuels in diesel engines for automotive or aviation.

The respiratory quotient (RQ), defined as the molar ratio between the produced carbon dioxide (CO₂) and the consumed oxygen (O₂), is a known and studied parameter mainly in the fermentation processes carried out in the presence of microaerophilic, aerobic, or anaerobic organism.

### BACKGROUND

For example, Bideaux C. et al., in “Applied and Environmental Microbiology” (2006), Vol. 72, No. 3, p. 2134-2140, report how to minimize glycerol production during a fermentation process for the production of ethanol from yeast Saccharomyces Cerevisiae using a metabolic model as a means of control. In particular, by monitoring the received feed during the cultivation of the Saccharomyces Cerevisiae yeast, the respiratory quotient (RQ) [defined as the ratio between the production of carbon dioxide (CO₂) and the consumed of oxygen (O₂)] is maintained at a value between 4 and 5. Compared to previous fermentation processes, wherein the feed glucose was not monitored, the final glycerol concentration was found to be decreased.

Heyman B. et al., in “Microbial Cell Factories” (2019), doi.org/10.1186/s12934-019-1126-9, report the respiratory quotient (RQ) as a control parameter for optimal oxygen delivery in production of 2,3-butanediol by fermentation of the bacterium Bacillus licheniformis DSM 8785, under microaerobic conditions. The combination of respiratory quotient (RQ) online monitoring with offline sampling provides a simple experimental route to determine the maximal concentration of 2,3-butanediol as 2,3-butanediol is consumed after glucose depletion. In this way, erroneous conclusions that can result from an offset between the sampling times and the time of the maximum concentration of 2,3-butanediol are avoided.

Xu J. et al., in “PNAS” (2017), Vol. 114 (27), E5308-E5316, report the production of lipids using dilute acetic acid, as such or in the form of salts, as a carbon source in the fermentation of Yarrowia lipolytica MTYL065 oleaginous yeast in a semi-continuous system. The supply of acetic acid and nitrogen is controlled using models based on metabolic methods and online measurements of the respiratory quotient.

Stockmann C. et al., in “FEMS Yeast Research” (2003), Vol. 4, p. 195-205, report online measurement of oxygen transfer rate (OTR) and carbon dioxide transfer rate (CTR) for the purpose of screening and the stabilization of mutants of Hansenual polymorpha. Initially, the oxygen transfer rate (OTR) increases exponentially and correlates with the exponential growth of the mutants. At the same time, the simultaneous growth of the carbon dioxide transfer rate (CTR) takes place, obtaining a value of the respiratory quotient (RQ) of about 1, indicative of the aerobic glucose turnover.

Ochoa-Estopier C. et al., in “Journal of Biotechnology” (2014), Vol. 170, p. 35-41, report the use of the respiratory quotient (RQ) to study the lipidogenic metabolism of the yeast Yarrowia lipolytica. For the aforementioned purpose, said yeast is grown in a chemostat and the key parameters are verified which highlight the passage from oxidative metabolism to the production of lipids: in particular, there is a respiratory quotient (RQ) equal to 1.04 when said yeast grows on glucose with a biomass yield equal to 0.47, while when the supply of the nitrogen source is reduced there is an increase in the respiratory quotient (RQ) indicates of the accumulation in the yeast cells of a more reduced compound compared to glucose (e.g. example, an accumulation of lipids).

It is also known that aerobic fermentations for the production of lipids are generally carried out by linking the parameter of dissolved oxygen (dO₂) to the stirring speed, in particular, the dissolved oxygen value (dO₂) is kept constant at 20%-30% of the saturation value, through variations in the stirring speed as reported, for example, by Capusoni C. et al., in “Bioresource Technology” (2017), Vol. 217, p. 281-289. However, this method is characterized by two critical points:
it does not provide direct indications of the metabolic activity in progress;
turns out to be ineffective under limiting oxygen conditions, i.e. in the conditions in which the speed with which oxygen (O₂) passes into solution (Oxygen Transfer Rate-OTR) and that with which it is absorbed by the microorganism used in fermentation (Oxygen Uptake Rate-OUR) are equal and the percentage of dissolved oxygen (dO₂) is close to zero.

### SUMMARY

The Applicant has therefore faced the problem of identifying, in fermentation processes for the production of lipids from oleaginous yeasts, a suitable parameter for the purpose of monitoring and controlling the cellular respiration of the oleaginous yeasts used, even in conditions of limiting oxygen, or in those conditions in which the high rate of growth leads to having a percentage of dissolved oxygen (dO₂) close to zero, despite the increase in the stirring rate and the continuous supply of air to the bioreactor [equal, for example, to 1 vvm (volume of air flowing per volume of culture medium per minute)].

The Applicant has now found that the cellular respiration of oleaginous yeasts used in fermentation processes can be controlled by monitoring the respiratory quotient (RQ) of said oleaginous yeasts. In particular, the Applicant has found a fermentation process for the production of lipids in the presence of at least one oleaginous yeast comprising: a first step of growth of the oleaginous cell biomass and a second step of lipid production, wherein the respiratory quotient (RQ) of said at least one oleaginous yeast is maintained at specific values, said values being different for said first step of growth of the oleaginous cell biomass and for said second step of lipid production and being kept constant during said first step and said second step.

More in particular, the Applicant has found that the monitoring of the respiratory quotient (RQ), defined as the molar ratio between the production of carbon dioxide (CO₂) and the consumed oxygen (O₂), allows to divide the above fermentation process in two steps, i.e.:
a first step of growth of the cell biomass;
a second step of lipid production.

During the first step, the growth of the cell biomass exclusively exploits the primary metabolism of the used oleaginous yeast. In agreement with the stoichiometric model also used by Mewa-Ngongang M. et al . . . , in “Fermentation” (2021), Vol. 7, 89, doi. org/10.3390/fermentation7020089, it is possible to define said first step according to the following equation:

$$0.25 {C}_{6} {H}_{12} {O}_{6} + 0.45 {O}_{2} + 0.2 N {H}_{4}^{+} = 1 C {H}_{18} {O}_{0.5} {N}_{0.2} + 0.9 {H}_{2} O + 0.5 C {O}_{2} + 0.2 {H}^{+} .$$

According to the above equation, in the first step of growth of the cell biomass (CH_{1,8}O_{0,5}N_{0,2}) from glucose (C₆H₁₂O₆), the respiratory quotient (RQ), i.e. the molar ratio between the produced carbon dioxide (CO₂) and the consumed oxygen (O₂) is equal to 1.1.

The second step of lipid production, triggered by a nutrient deficiency, in particular nutrient depletion, more particularly nitrogen depletion, as reported, for example, by Galafassi S. et al., in “Bioresource Technology” (2012), Vol. 111, p. 398-403, is characterized by a higher respiratory quotient (RQ). Said second step can be defined according to the following equation as reported, for example, by Ratledge C. et al., in “Progress in Industrial Microbiology” (1982), Vol. 16, p. 119-206:

$$16 {C}_{6} {H}_{12} {O}_{6} + 15 {O}_{2} + 0.2 {NH}_{4}^{+} = 1 C {H}_{57} {H}_{104} {O}_{6} + C {H}_{1.8} {O}_{0.5} {N}_{0.2} + 43.5 {H}_{2} O + 38.1 {CO}_{2} + 0.2 {H}^{+} .$$

Considering a production of lipids consisting, for simplification, of triolein alone (CH₅₇H₁₀₄O₆), the result is a respiratory quotient (RQ) equal to 2.54.

The lipids thus obtained can be advantageously used in the production of biofuels which can be used as such, or mixed with other fuels in diesel engines for automotive or aviation.

The present disclosure therefore provides a fermentation process for the production of lipids in the presence of at least one oleaginous yeast comprising:
a first step of growth of the oleaginous cell biomass wherein the respiratory quotient (RQ) of said at least one oleaginous yeast is kept at a constant value comprised between 0.85 and 1.2, preferably comprised between 0.9 and 1.15, more preferably equal to 1.1;
a second step of lipid production wherein the respiratory quotient (RQ) of said at least one oleaginous yeast is kept at a constant value comprised between 1.2 and 2.6, preferably comprised between 1.3 and 2.55, more preferably equal to 1.4.

For the purpose of the present description and of the following claims, the definitions of the numerical ranges always include the extremes unless otherwise specified.

For the purposes of the present description and of the following claims, the term “comprising” also includes the terms “consisting essentially of” or “consisting of”.

According to a preferred embodiment of the present disclosure, said respiratory quotient (RQ) can be monitored by “off-gas” analysis.

According to a preferred embodiment of the present disclosure, said respiratory quotient (RQ) can be controlled by feeding oxygen to the culture medium where the fermentation takes place, preferably by adjusting the stirring speed, the over-pressure or the oxygen concentration in the incoming air (i.e. air fed to the culture medium), more preferably by adjusting the stirring speed. 25 According to a preferred embodiment of the present disclosure, said oleaginous yeast can be selected from: Rhodosporidium azoricum, Trichosporon pullulans, Trichosporon oleaginous, Trichosporon cacaoliposimilis, Cryptococcus curvatus, Rhodotorula gracilis, Rhodotorula graminis, Lypomices starkeyi, Lypomices lipofer, Trigonopsis variabilis, Candida kefyr, Candida curvata, Candida lipolytica, Torulopsis sp., Pichia stipitis.

According to a particularly preferred embodiment of the present disclosure, said oleaginous yeast can be selected from: Rhodosporidium azoricum DSM 29495 (mutant described in patent application WO 2016/108185), Trichosporon pullulans NRRL Y-1522 (commercially available strain), Trichosporon oleaginous ATCC 20509 (commercially available strain), preferably is Rhodosporidium azoricum DSM 29495.

According to a preferred embodiment of the present disclosure, said first step can be carried out at a temperature comprised between 20° C. and 40° C., preferably comprised between 25° C. and 35° C.

According to a preferred embodiment of the present disclosure, said first step can be carried out for a time comprised between 20 hours and 60 hours, preferably comprised between 25 hours and 50 hours.

According to a preferred embodiment of the present disclosure, said first step can be carried out at a stirring speed comprised between 600 rpm and 1100 rpm, preferably comprised between 650 rpm and 1050 rpm.

According to a preferred embodiment of the present disclosure, said second step can be carried out at a temperature comprised between 20° C. and 40° C., preferably comprised between 25° C. and 35° C.

According to a preferred embodiment of the present disclosure, said second step can be carried out for a time comprised between 20 hours and 60 hours, preferably comprised between 25 hours and 50 hours.

According to a preferred embodiment of the present disclosure, said second step can be carried out at a stirring speed comprised between 850 rpm and 1150 rpm, preferably comprised between 900 rpm and 1100 rpm.

According to a preferred embodiment of the present disclosure, said fermentation can be carried out at a pH comprised between 4.5 and 7, preferably comprised between 5 and 6.7. In order to maintain the pH in the desired ranges, an aqueous solution of at least one inorganic base can be added to the culture medium used for fermentation, such as, for example, ammonium hydroxide, sodium hydroxide, potassium hydroxide, calcium hydroxide, magnesium hydroxide, or mixtures thereof, preferably potassium hydroxide; or an aqueous solution of at least one inorganic acid such as, for example, phosphoric acid, sulfuric acid, 2-(N-morpholino) ethane sulfonic acid (MES), hydrochloric acid, or mixtures thereof; in such quantity as to obtain the desired pH. Preferably, an aqueous solution of 2-(N-morpholino) ethane sulfonic acid (MES) can be added.

According to a preferred embodiment of the present disclosure, said fermentation is a fermentation in batch, or in discontinuous culture (fed-batch fermentation), or in continuous culture, preferably in batch, or in discontinuous culture (fed-batch fermentation).

For the purpose of the present disclosure, said fermentation can be carried out in a reaction device with internal circulation of the “Air-Lift” type described, for example, in international patent application WO 2017/046720, or in a mechanically stirred reactor.

Preferably, said oleaginous yeast, before being used in said fermentation, can be grown in a culture medium comprising xylose, cellobiose, glucose, or mixtures thereof, at a concentration preferably comprised between 1% by weight and 3% by weight with respect to the total weight of said culture medium. Said fermentation can advantageously be carried out in fermentation devices known in the art, in the presence of culture media comprising various nutrients such as, for example, nitrogen, potassium phosphate, magnesium, salts, vitamins.

In said fermentation device, the fermentation can be carried out in the presence of culture media comprising sugars with 6 carbon atoms (C6), mainly glucose, sugars with 5 carbon atoms (C5), mainly xylose, various nutrients such as, for example, nitrogen, potassium phosphate, magnesium, salts, vitamins, microelements, normally used in culture media.

Alternatively, in said fermentation device, as a source of sugars with 6 carbon atoms (C6) and with 5 carbon atoms (C5), a hydrolysate deriving from lignocellulosic biomass can be used. For this purpose, various lignocellulosic biomasses can be used: a detailed description of the lignocellulosic biomasses which can be advantageously used can be found, for example, in the international patent application WO 2015/028156, incorporated herein by reference. Preferably, said lignocellulosic biomass can be selected, for example, from:
plants specifically cultivated for energy use such as, for example, miscanthus, panicum (Panicum virgatum), common reed (Arundo donax);
plants not cultivated specifically for energy use such as, for example, sorghum (for example, sorghum fibres);
scraps, residues and waste of agricultural products such as, for example, guayule, corn (for example, corn stalks, corn cobs), soybeans, cotton, flax, rapeseed, wheat (for example, wheat straw), rice (for example, rice straw, rice hull, rice husk), sugarcane (for example, sugarcane straw, sugarcane bagasse), palm (for example, palm leafs, palm trunks, palm mibrids, palm empty fruit bunches);
scraps, residues and waste of products deriving from forestation, forestry, or woodworking such as, for example, poplar, alder, birch;
scraps from agro-food products intended for human consumption or zootechnics;
chemically untreated residues from the paper industry;
waste from separate collection of municipal solid waste (for example, urban waste of vegetable origin, paper);
algae such as, for example, microalgae or macroalgae, in particular macroalgae.

At the end of the fermentation, in order to deactivate the lipolytic enzymes (e.g., lipase), the fermentation broth obtained can be subjected to heat treatment, preferably in the presence of sulfuric acid. Said heat treatment can be carried out at a temperature comprised between 70° C. and 120° C., preferably comprised between 75° C. and 110° C., for a time comprised between 5 minutes and 8 hours, preferably comprised between 2 hours and 4 hours. In the case of thermal treatment in the presence of sulfuric acid, the pH of the obtained aqueous suspension of oleaginous cell biomass comprising lipids can be comprised between 1.5 and 6.0, preferably between 2.0 and 4.5: more details on said heat treatment in the presence of sulfuric acid can be found, for example, in the international patent application WO 2017/021931.

At the end of the fermentation (i.e. first step and second step) the separation to which said fermentation broth is subjected in order to recover an aqueous suspension of oleaginous cell biomass comprising lipids and an aqueous phase (said aqueous phase optionally comprising suspended solids, for example, cells of the oleaginous microorganism used in the fermentation, or particulate deriving from the deterioration of the equipment used in the process, or from the precipitation of salts), can be implemented through methods known in the art such as, for example, filtration, filter pressing, microfiltration or ultrafiltration, centrifugation.

In order to further concentrate the aqueous suspension of oleaginous cell biomass comprising lipids obtained after separation, said aqueous suspension of oleaginous cell biomass, before being subjected to lipid recovery (i.e. cell lysis, solvent extraction and solvent evaporation), can be subjected to centrifugation. Said centrifugation can be carried out for a time comprised between 5 minutes and 30 minutes, preferably comprised between 15 minutes and 25 minutes, at a rotation speed comprised between 3000 rpm and 9000 rpm, preferably comprised between 3500 rpm and 8000 rpm.

The concentration of the oleaginous cell biomass obtained can be measured in grams per liter of fermentation broth, by determining the dry weight of the oleaginous yeast cells of a sample of fermentation broth of known volume taken at predetermined intervals and at the end of the fermentation. In particular, dry weight of the oleaginous cell biomass means the weight of the cells contained in a known volume of fermentation broth, determined by weighing the aforementioned cells after having eliminated all the water content by filtration on Whatman GF/F filters (0.7 μm) and subsequent heat treatment in a ventilated stove at 105° C. until constant weight (about 24 hours).

In order to recover the lipids, said aqueous suspension of oleaginous cell biomass comprising lipids can be subjected to cell lysis, which can be carried out by various methods. Non-limiting examples of these methods are:
heat treatment, which can be carried out using pressurized autoclaves (for example, Brignole autoclave Mod. AU-2, or Parr stirred reactor Mod. PA 4575), at a pressure comprised between 2 bar and 6.5 bar, preferably comprised between 3 bar and 5.5 bar, at a temperature comprised between 100° C. and 160° C., preferably comprised between 110° C. and 150° C., for a time comprised between 1 hour and 8 hours, preferably comprised between 1.5 hours and 4 hours, under stirring comprised between 100 rpm and 800 rpm, preferably comprised between 400 rpm and 600 rpm, as described, for example, in international patent application WO 2012/052368;
mechanical treatment, which can be carried out using high pressure homogenizers (for example, homogenizer Mod. NS3006L by Gea NiroSoavi), at a pressure comprised between 800 bar and 2000 bar, preferably comprised between 1000 bar and 1600 bar, at a temperature comprised between 10° C. and 100° C., preferably comprised between 20° C. and 80° C., at a flow rate of the aqueous suspension of oleaginous cell biomass comprised between 5 l/h and 50 l/h, preferably comprised between 7 l/h and 40 l/h;
microwave treatment, which can be carried out using microwave equipment (for example, microwave apparatus Mod. MycroSYNTH by Milestone), at a temperature comprised between 45° C. and 150° C., preferably comprised between 50° C. and 100° C., for a time comprised between 10 minutes and 2 hours, preferably comprised between 15 minutes and 1 hour.

At the end of said cell lysis, the lipids can be recovered from the aqueous suspension of exhausted cell biomass comprising lipids obtained, by extraction using, for example, a reflux extractor.

Said extraction can be carried out in the presence of at least one organic solvent which can be selected, for example, from: apolar organic solvents such as, for example, iso-octane, n-octane, n-heptane, or mixtures thereof; mixtures of hydrocarbons such as, for example, naphtha or diesel cuts which may possibly also derive from the production of renewable fuels for diesel or aviation engines; polar organic solvents such as, for example, methanol, ethanol, iso-propanol, acetone, ethyl acetate, hexane, methyl-tert-butyl ketone, ethyl-tert-butyl ether, or mixtures thereof; or mixtures thereof.

Said extraction can be carried out at a temperature comprised between 20° C. and 200° C., preferably at the boiling temperature of the solvent used.

Said extraction can be carried out in the presence of an amount of solvent comprised between 1 and 6 times, preferably comprised between 1.5 times and 5 times, the volume of the aqueous phase of the aqueous suspension of exhausted oleaginous cell biomass comprising lipids obtained from cell lysis.

The aqueous suspension of spent oleaginous cell biomass comprising lipids obtained after said cell lysis can be subjected to extraction one or more times. Preferably, said aqueous suspension of spent oleaginous cell biomass comprising lipids can be subjected to extraction from 1 time to 5 times, more preferably from 1 time to 3 times.

At the end of the aforementioned extraction, the following two phases are obtained:
(i) an organic phase comprising lipids dissolved in solvent;
(ii) an aqueous phase comprising cellular debris and traces of unseparated lipids.

In order to recover the lipids, said organic phase (i) is subjected to evaporation, obtaining as a residue a high-boiling oil (ia) comprising lipids and a liquid phase containing the solvent which can be recycled to the above extraction.

Preferably, the lipids included in said organic phase (i) are triglycerides, more preferably esters of glycerol with fatty acids having from 14 to 24 carbon atoms such as, for example, palmitic acid, stearic acid, oleic acid, α-linoleic acid, in an amount greater than or equal to 80% by weight, preferably greater than or equal to 90% by weight, with respect to the total weight of the lipids. Other lipids which may be present in said organic phase (i) are: phospholipids, monoglycerides, diglycerides, free fatty acids, or mixtures thereof.

The total amount of lipids present in the aqueous suspension of oleaginous cell biomass obtained after fermentation, as well as the total amount of lipids contained in said high boiling oil (ia), can be determined by methods known in the art such as, for example, the colorimetric method which is based on the reaction of lipids with phosphoric acid and phosphovanillin using, for example, the “total lipids-sulpho-phospho vanilline” kit marketed by Spinreact Sa/SAU, Ctra Santa Coloma, 7 E-17176 Sant Esteve de Bas (GI), Spain. Further details of this method can be found, for example, in the following article: “Chemical Basis of the Sulpho-phospho-vanillin Reaction for Estimating Total Serum Lipids”, JA Knight et al., published in “Clinical Chemistry” (1972), Vol. 18, No. 3, p. 199-202.

Said aqueous phase (ii) comprising the cellular debris, in particular proteins and polysaccharides contained in the cell membrane of the oleaginous microorganism used, can be dehumidified and used as fuel.

Alternatively, said aqueous phase (ii) can be subjected to anaerobic digestion for the production of biogas, which can be used for the production of electricity, which can also be used to satisfy the energy requirement of the process of the present disclosure.

Alternatively, said aqueous phase (ii) can be subjected to liquefaction for the production of bio-oil as described, for example, in international patent applications WO 2010/069583 or WO 2010/069516.

The lipids obtained according to the process of the present disclosure can be advantageously used in the production of biofuels which can be used as such, or mixed with other fuels in diesel engines for automotive or aviation.

In order to better understand the present disclosure and to put it into practice, some illustrative and non-limiting examples of the same are given below.

### Example 1 (Disclosure)

Two-step fermentation at constant respiratory quotient (RQ).

For this purpose, a suspension of cells of Rhodosporidium azoricum DSM 29495 (pre-inoculation) was prepared by operating as follows.

20 μl of Rhodosporidium azoricum DSM 29495 cells (20% glycerol), stored at −80° C., were inoculated in 20 ml of YEPD-medium composed of glucose (20 g/L), yeast extract (10 g/L) and peptone (20 g/L) to obtain a cell suspension which was kept at 30° C., under stirring (200 rpm), for 24 hours.

At the end of the 24 hours, the obtained suspension of Rhodosporidium azoricum DSM 29495 cells (pre-inoculation), was inoculated in a 1 liter fermenter (Biostat®B by Sartorius Stedim) equipped with an off-gas analysis system (BlueInOne Ferm by BlueSens), operating under the following conditions:
culture medium: 100 g/L glucose, 5.82 g/L (NH₄)₂HPO₄, 0.03 g/L of MgSO_{4.7}H₂O, 0.06 g/L of NaCl, 0.06 g/L of CaCl₂·2H₂O, 2.47 g/L KOH, 2.16 g/LH₂SO₄, 30 g/L corn steep liquor;
volume: 600 ml;
pH: 5, maintained by adding an aqueous solution of NH₄OH at 13.5% by volume (v/v) for the first 24 hours and, subsequently by adding KOH 2.5 M;
feeding of a further 30 g/L corn steep liquor after 20 hours;
feeding a concentrated aqueous solution of glucose at 50% by weight (w/w) with variable feed flow in order to keep the glucose concentration constant at 30 g/L;
inlet air flow: 0.7 L/minute;
temperature: 30° C. (same in the first step and in the second step);
variable stirring comprised between 700 rpm and 1040 rpm in the first step and between 950 rpm and 1050 rpm in the second step;
total fermentation duration: 96 hours.

Starting from 26 hours (first step of biomass growth) a condition of limiting oxygen (O₂) was triggered characterized by a percentage of oxygen (pO₂) equal to zero as shown in

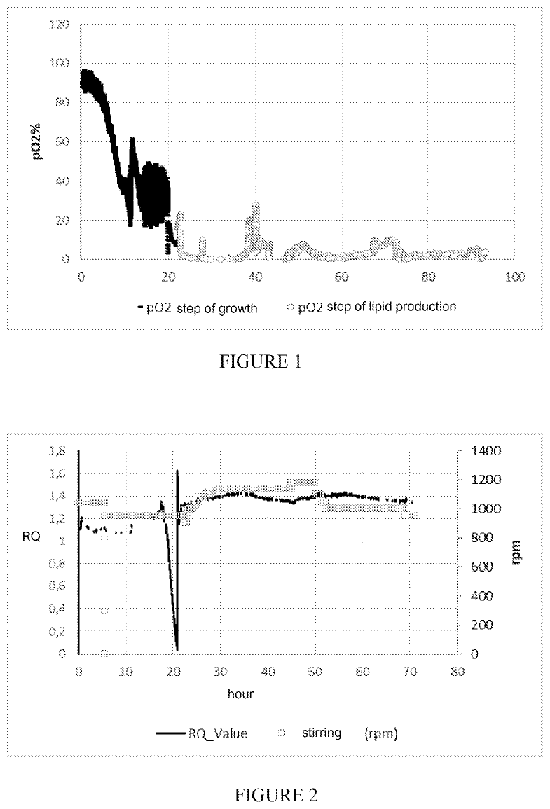

FIG. 1 [the abscissa shows the first step (pO₂ growth step) (hours) and the second step (pO₂ lipid production step) (hours), the percentage of oxygen (pO₂) (%) is reported in the ordinate].

It was possible through the analysis of the off-gas to calculate the respiratory quotient (RQ), i.e. the molar ratio between the produced carbon dioxide (CO₂) and the consumed oxygen (O₂), and to divide the fermentation in two steps.

During the first 26 hours (first step of biomass growth), 54.15 g/L of cell biomass (dry weight) (Cell Dry Weight-CDW) was produced with a yield, with respect to the glucose consumed (59,9 g), equal to 0.44 g/g: in this step a respiratory quotient (RQ) equal to 1.1 was recorded, highlighting the preponderance of the primary metabolism.

The addition of a source of nitrogen (corn steep liquor) at 20 hours, thanks to the regulation of the stirring (i.e. stirring comprised between 700 rpm and 1040 rpm) while leading to a further growth from the cell biomass did not lead to an increase in respiratory quotient (RQ) which was kept at 1.1.

The second step of lipid production begins after 46 hours: in said second step, thanks to the regulation of the stirring (i.e. stirring between 950 rpm and 1050 rpm) the respiratory quotient (RQ) was kept constant at a value equal to 1.4 obtaining the production of 64.2 g/L of lipids (total production of the entire fermentation cycle, ie first step+second step).

Considering the lipids produced at the end of the entire fermentation cycle (i.e. 64.2 g/L) compared to the glucose consumed (261.3 g), a yield equal to 0.19 g/g was obtained, while considering only the lipids (i.e. 39.8 g/L) produced in the second step with a constant respiratory quotient (RQ), a yield of lipids with respect to the consumed glucose (118.3 g) equal to 0.34 g/g was obtained.

FIG. 2 shows the profile of the respiratory quotient (RQ) (in the left ordinate) and of the stirring speed (rpm) (in the right ordinate) and the time (hours) (in the abscissa) for the second step.

At the end of the fermentation, a sample of fermentation broth (5 ml) was taken and subjected to centrifugation for 10 minutes, at 4000 rpm, obtaining an aqueous suspension of concentrated oleaginous cell biomass comprising lipids and an aqueous phase. Said aqueous suspension of concentrated oleaginous cell biomass comprising lipids was used for the determination of dry weight (by filtration and subsequent heat treatment) and of lipids (using the total lipids-sulpho-phospho vanilline kit), operating as described above.

### EXAMPLE 2 (COMPARATIVE)

Two-Step Fermentation with Variable Respiratory Quotient (RQ).

A suspension of cells of Rhodosporidium azoricum DSM 29495 obtained as described in Example 1 (pre-inoculation), was inoculated in a 1 liter fermenter (Biostat®B by Sartorius Stedim) equipped with an off-gas analysis system (BlueInOne Ferm by BlueSens), operating under the following conditions:
culture medium: 100 g/L glucose, 5.82 g/L (NH₄)₂HPO₄, 0.03 g/L of MgSO_{4.7}H₂O, 0.06 g/L of NaCl, 0.06 g/L of CaCl₂)·2H₂O, 2.47 g/L KOH, 2.16 g/LH₂SO₄, 30 g/L corn steep liquor;
volume: 600 ml;
pH: 5, maintained by adding an aqueous solution of NH₄OH at 13.5% by volume (v/v) for the first 24 hours and, subsequently by adding KOH 2.5 M; feeding a further 30 g/L corn steep liquor after 20 hours;—feeding a concentrated aqueous solution of glucose at 50% by weight (w/w) with variable feed flow in order to keep the glucose concentration constant at 30 g/l;
inlet air flow: 0.7 L/minute;
temperature: 30° C. (same in the first step and in the second step);
variable stirring between 700 rpm and 1040 rpm in the first step and between 950 rpm and 1050 rpm in the second step; said stirring was automatically adjusted in order to maintain the dissolved oxygen value (dO₂) equal to 30% of the saturation value and, in the second step, when the limiting oxygen condition occurred (percentage of dissolved oxygen (dO₂) equal to zero) was kept stable at the set maximum value (i.e. 1050 rpm);
total fermentation duration: 96 hours.

It was possible through the analysis of the off-gas to calculate the respiratory quotient (RQ), i.e. the molar ratio between the produced carbon dioxide (CO₂) and the consumed oxygen (O₂), which resulted to be variable during the duration of fermentation and between 0.8 and 1.4.

During the first 26 hours (first step of biomass growth), 60.45 g/L of cell biomass (dry weight) (Cell Dry Weight-CDW) were produced with a yield, with respect to the consumed glucose (94, 3 g), equal to 0.45 g/g: in this step a variable respiratory quotient (RQ) between 0.8 and 1.1 was recorded, highlighting the preponderance of the primary metabolism. In said first step 5 g/L of lipids were produced with respect to the glucose consumed (94.3 g) with a yield equal to 0.03 g/g.

The addition of a nitrogen source (corn steep liquor) at 20 hours led to a further growth of the cell biomass with a consequent increase in the respiratory quotient (RQ) which resulted to be equal to 1.2 and to a decrease in growth rate and metabolism as shown in

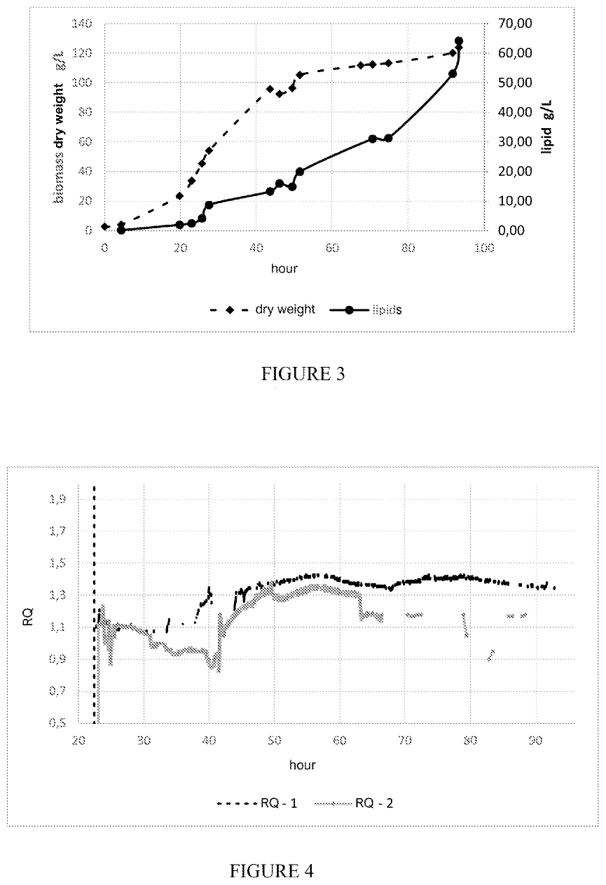

FIG. 3 [the cell biomass (dry weight) (Cell Dry Weight-CDW) is shown in the right ordinate, the amount of lipids produced in the fermentation is shown in the left ordinate (first step+second step) (g/L), the time (hours) is shown on the abscissa].

The second step of lipid production begins after 46 hours: in said second step a variable respiratory quotient (RQ) between 0.7 and 1.4 was recorded, resulting in the production of 15.68 g/L of lipids (total production of the entire fermentation cycle, ie first step+second step).

Considering the lipids produced at the end of the entire fermentation cycle (i.e. 15.68 g/L) compared to the glucose consumed (313.6 g), a yield equal to 0.05 g/g was obtained, while considering only the lipids produced in the second step at a variable respiratory quotient (RQ) (12.02 g/L), a yield of lipids with respect to the consumed glucose (112 g) equal to 0.11 g/g was obtained.

The data reported in Example 2 (comparative) clearly indicate that a lower production of lipids is obtained when the fermentation is carried out at variable respiratory quotient values.

In FIG. 4 [the abscissa shows the time in hours (h), the ordinate shows the respiratory quotient (RQ)] the profiles of the respiratory quotient (RQ) recorded during the fermentation of Example 1 (disclosure) (RQ-1) and Example 2 (comparative) (RQ-2) are reported.

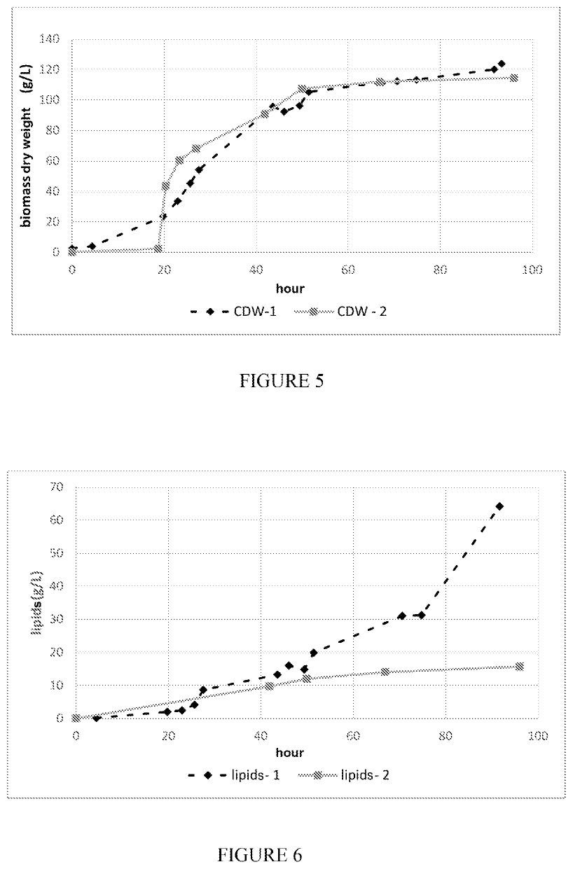

In FIG. 5 [the abscissa shows the time in hours (h), the ordinate shows the dry weight (Cell Dry Weight-CDW) expressed in grams/liter (g/L)] the dry weight profiles (Cell Dry Weight-CDW) recorded during the fermentation of Example 1 (disclosure) (CDW-1) and Example 2 (comparative) (CDW-2) are reported.

In FIG. 6 [the abscissa shows the time in hours (h), the ordinate shows the concentration of lipids expressed in grams/liter (g/L)] the profiles of the lipid concentration recorded during fermentation (total fermentation, i.e. 1st step+2nd step) of Example 1 (disclosure) (lipids-1) and of Example 2 (comparative) (lipids-2) are reported.

1. A fermentation process for the production of lipids in the presence of at least one oleaginous yeast, the process comprising the following steps:
a first step of growth of the oleaginous cell biomass wherein the respiratory quotient (RQ) of said at least one oleaginous yeast is kept at a constant value comprised between 0.85 and 1.2; and
a second step of lipid production wherein the respiratory quotient (RQ) of said at least one oleaginous yeast is kept at a constant value comprised between 1.2 and 2.6.

2. The fermentation process for the production of lipids according to claim 1, wherein said respiratory quotient (RQ) is monitored by an off-gas analysis.

3. The fermentation process for the production of lipids according to claim 1, wherein said respiratory quotient (RQ) is controlled by feeding oxygen to the culture medium where the fermentation takes place, by adjusting the stirring speed, the over-pressure or oxygen concentration in the incoming air (i.e. air fed to the culture medium.

4. The fermentation process for the production of lipids according to claim 1, wherein said oleaginous yeast is selected from the group consisting of: Rhodosporidium azoricum, Trichosporon pullulans, Trichosporon oleaginous, Trichosporon cacaoliposimilis, Cryptococcus curvatus, Rhodotorula gracilis, Rhodotorula graminis, Lypomices starkeyi, Lypomices lipofer, Trigonopsis variabilis, Candida kefyr, Candida curvata, Candida lipolytica, Torulopsis sp., and Pichia stipitis.

5. The fermentation process for the production of lipids according to claim 1, wherein said oleaginous yeast is selected from the group consisting of: Rhodosporidium azoricum DSM 29495, Trichosporon pullulans NRRL Y-1522, and Trichosporon oleaginous ATCC 20509.

6. The fermentation process for the production of lipids according to claim 1, wherein said first step is carried out:
at a temperature comprised between 20° C. and 40° C.; and/or
for a time comprised between 20 and 60 hours; and/or
at a stirring speed comprised between 600 rpm and 1100 rpm.

7. The fermentation process for the production of lipids according to claim 1, wherein said second step is carried out:
at a temperature comprised between 20° C. and 40° C.; and/or
for a time comprised between 20 and 60 hours; and/or
at a stirring speed comprised between 850 rpm and 1150 rpm.

8. The fermentation process for the production of lipids according to claim 1, wherein said fermentation is carried out at a pH comprised between 4.5 and 7, preferably comprised between 5 and 6.7.

9. The fermentation process for the production of lipids according to claim 1, wherein said fermentation is a fermentation in batch, in discontinuous culture (fed-batch fermentation), or in continuous culture, preferably in batch, or in discontinuous culture (fed batch fermentation).
